(19) United States
(12) Patent Application Publication
(10) Pub. No.: US 2008/0305143 A1
(43) Pub. Date: Dec. 11, 2008

(54) Controlled Radical Polymerization-Derived Block Copolymer Compositions for Medical Device Coatings

(75) Inventors: Mingfei Chen, Santa Rosa, CA (US); Peiwen Cheng, Santa Rosa, CA (US); Kishore Udipi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/064,110

(22) PCT Filed: Aug. 9, 2006
(86) PCT No.: PCT/US06/31289

§ 371 Date: Feb. 19, 2008

Related U.S. Application Data
(60) Provisional application No. 60/711,800, filed on Aug. 25, 2005.

(51) Int. Cl. A61F 2/02; A61L 27/34

(52) U.S. Cl. 424/423; 427/2.25

(57) ABSTRACT

Controlled radical polymerization-derived biocompatible block copolymer coatings for medical devices are disclosed. Specifically, block copolymer coatings designed to control the release of bioactive agents from medical devices in vivo are disclosed. The present application also discloses providing vascular stents with drug-eluting controlled release block copolymer coatings and related methods for making these medical devices and coatings.

### FIELD OF THE INVENTION

This invention relates generally to controlled radical polymerization-derived block copolymer compositions useful as biocompatible coatings for medical devices. More specifically, the present invention relates to block copolymer coatings designed to control the release of bioactive agents from a medical device. Even more specifically the present invention relates to providing vascular stents with controlled release coatings made of block copolymers and related methods for making these coatings.

### BACKGROUND OF THE INVENTION

Medical devices are used for myriad purposes on and throughout an animal's body. They can be simple ex vivo devices such as adhesive bandages, canes, walkers and contact lenses or complex implantable devices including pacemakers, heart valves, vascular stents, catheters and vascular grafts. Implantable medical devices must be biocompatible to prevent inducing life threatening adverse physiological responses between the implant recipient and device.

Recently, highly biocompatible polymers have been formulated to provide implantable medical devices with coatings. These coatings not only increase an implant's tissue compatibility but can also function as bioactive agent reservoirs. However, designing polymer coatings for medical devices has proven problematic. All medical device coatings must be non-toxic, durable and adhere well to device surfaces. Additionally, when the medical device comes into intimate contact with unprotected tissues such as blood and internal organs it must also be biocompatible. Furthermore, if the medical device is designed to be pliable either in operation or deployment, the coating must resist cracking, fracture and delamination.

Moreover, medical devices intended to act as bioactive agent (drug) reservoirs must not only be biocompatible, structurally stable and resistant to delamination, but also chemically compatible with the drug to be deployed. Furthermore, if the reservoir is also intended to control the drug's release rate into adjacent tissue the polymer used must possess other highly specialized properties as well.

One of the most widely used techniques to modify the properties of a polymer material is to blend different homopolymers or copolymers together into a single mixture. The resulting polymer mixture hypothetically possess a combination of properties inherent in each polymer or copolymer component of the blend. However, not all polymers are miscible and thus instead of forming a uniform blend, the polymers form immiscible mixtures subject to phase separation and delamination. When used as coatings for medical devices this problem becomes even more pronounced. One polymer component may have a stronger affinity for the medical device surface than another and thus may layer closer to the medical device surface. The polymer component having less affinity and avidity for the medical device surface migrates away from the medical device surface resulting in a bi-layer where each polymer component retains its individual properties and the coating no longer functions as a cohesive uniform substance. When bioactive agents are included in the mixture, the problems associated with immiscibility are magnified by the addition of yet a third chemical species having unique chemical properties. Thus prior art methods used to develop polymer coatings, specifically drug-eluting coatings, has been largely by trial and error. Recently, the present inventors have developed methods for reducing uncertainty in coating design by matching polymer components with bioactive agents based in part on solubility factors, see for example co-pending U.S. patent application Ser. No. 11/005,463. While these procedures have significantly advanced polymer coating science, the primary focus of the '463 application is directed at polymer blends not block copolymers.

Block copolymers may be potentially important polymer compositions for use as medical device coatings and as drug-eluting reservoirs. Block copolymers are not blends, but rather copolymers having individual subunits integrated into a single macromolecule. Consequently these are stable compounds not prone to delaminate or separate. Moreover, pendent R groups present within each block can be modified to increase or decrease overall polymer miscibility with bioactive agents without adversely affecting the polymer's structural performance characteristics. Unfortunately, block copolymers are very difficult to synthesize and thus there are only a limited number of polymers commercially available for medical use. However, recent advances in synthetic chemistry has led to the development of new methods for free radical polymerization-specifically atom transfer radical polymerization (ATRP) and reversible addition-fragmentation chain transfer (RAFT). These new synthetic methods can provide for the convenient synthesis of a wide range of block copolymers that were previously impossible or difficult to make.

U.S. Pat. No. 6,855,770 B2 (hereinafter the '770 patent) issued Feb. 15, 2005 to Pinchuck et al. describe certain medical grade block copolymers useful for drug delivery. The '770 patent discloses a block copolymer comprising one or more elastomeric blocks and one or more thermoplastic blocks combined with a therapeutic agent, specifically a polystyrene-polyisobutylene-polystyrene copolymer combined with paclitaxel and used to coat a vascular stent. The block copolymers in the '770 patent are made using carbocationic polymerization (living ionic polymerization) and is conducted under conditions that minimize or avoid chain transfer termination of the growing chain. However, these prior art methods are very susceptible to attack, and thus termination, by active hydrogens; consequently water, alcohol and the like must be kept to a minimum. This inherent limitation in prior art methods significantly limits the range of solvents and hydrocarbons that can be used. These limited reaction conditions and monomer subunit selection lead to a narrow range of polymer types and thus restricted compatibility with diverse bioactive agents.

Therefore, it is an object of the present invention to provide methods for making a wide range of biocompatible polymers, and the polymers themselves, useful as drug-delivery medical devices.

### SUMMARY OF THE INVENTION

The present invention provides medical devices having controlled release drug-eluting coatings comprising block copolymers. The block copolymers of the present invention are synthesized using highly versatile living radical polymerization techniques—specifically new methods of living free radical polymerization including atom transfer radical polymerization (ATRP) and reversible addition-fragmentation chain transfer (RAFT). In particular, block copolymers made in accordance with the teachings of the present invention are suitable for drug delivery by vascular stents. The block copolymers can be customized to deliver hydrophilic or hydrophobic drugs or large molecules such as proteins or DNA (genes).

### BRIEF DESCRIPTION OF THE FIGURES

FIG. 1 graphically depicts idealized first-order kinetics associated with drug release from a polymer coating.

FIG. 2 graphically depicts idealized zero-order kinetics associated with drug release from a polymer coating.

FIG. 3 depicts a tortuous path tubing system used to test coating durability.

FIG. 4 depicts a medical device, specifically a vascular stent having the coating made in accordance with the teachings of the present invention thereon.

FIG. 5 a-d depict cross sections of the various coating configurations used to provide vascular stents with the controlled release coatings made in accordance with the teachings of the present invention.

FIG. 6 depicts a vascular stent having a coating made in accordance with the teachings of the present invention mounted on a suitable delivery device—a balloon catheter.

### DEFINITION OF TERMS

Prior to setting forth the invention, it may be helpful to an understanding thereof to set forth definitions of certain terms that will be used hereinafter:

Animal: As used herein “animal” shall include mammals, fish, reptiles and birds. Mammals include, but are not limited to, primates, including humans, dogs, cats, goats, sheep, rabbits, pigs, horses and cows.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Bioactive agent: As used herein “bioactive agent” shall included anti-proliferative compounds, cytostatic compounds, toxic compounds, anti-inflammatory compounds, analgesics, antibiotics, protease inhibitors, statins, nucleic acids, polypeptides, and delivery vectors including recombinant micro-organisms, liposomes, the like (see Drugs below).

Block copolymer: As used herein “block copolymer” a macromolecule composed of block (a portion of a macromolecule comprising many constitutional units [an atom or group of atoms, including pendant atoms or groups, if any]) comprising a part of the essential structure of a macromolecule, that has at least one feature which is not present in the adjacent portions wherein said “blocks” are arranged in a linear sequence.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first-order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero-order kinetics), that is the drug is released in even amounts over a predetermined time (with or without an initial burst phase) or may be a gradient release. A gradient release implies that the concentration of drug released from the device surface changes over time.

Copolymer: As used here in a “copolymer” will be defined as a macromolecule produced by the simultaneous or step-wise polymerization of two or more dissimilar units such as monomers. Copolymer shall include bipolymers (two dissimilar units), terpolymers (three dissimilar units), etc.

Drug(s): As used herein “drug” shall include any bioactive agent having a therapeutic effect in an animal. Exemplary, non limiting examples include anti-proliferatives including, but not limited to, macrolide antibiotics including FKBP 12 binding compounds; for example zotarolimus (the USAN for a tetrazole-containing rapamycin analogue formally referred to as ABT-578 as described in U.S. Pat. No. 6,015,815), estrogens, chaperone inhibitors, leptomycin B, protease inhibitors, protein-tyrosine kinase inhibitors, peroxisome proliferator-activated receptor gamma ligands (PPARy), hypothemycin, nitric oxide, bisphosphonates, epidermal growth factor inhibitors, antibodies, proteasome inhibitors, antibiotics, anti-inflammatories, anti-sense nucleotides and transforming nucleic acids.

Ductility: As used herein “ductility, or ductile” is a polymer attribute characterized by the polymer's resistance to fracture or cracking when folded, stressed or strained at operating temperatures. When used in reference to the polymer coating compostions of the present invention the normal operating temperature for the coating will be between room temperature and body temperature or approximately between 15° C. and 40° C. Polymer durability in a defined environment is often a function of its elasticity/ductility.

Glass Transition Point: As used herein “glass transition point” or “Tg” is the temperature at which an amorphous polymer becomes hard and brittle like glass. At temperatures above its Tg, a polymer is elastic or rubbery; at temperatures below its Tg the polymer is hard and brittle like glass. The Tg may be used as a predictive value for elasticity/ductility.

Hydrophillic: As used herein in reference to the bioactive agent, the term “hydrophilic” refers to a bioactive agent that has a solubility in water of more than 200 micrograms per milliliter.

Hydrophobic: As used herein in reference to the bioactive agent the term “hydrophobic” refers to a bioactive agent that has a solubility in water of no more than 200 micrograms per milliliter.

### DETAILED DESCRIPTION OF THE INVENTION

The present invention is directed at engineering block copolymers that provide optimized drug-eluting medical devices coatings. Specifically, block copolymers made in accordance with teachings of the present invention provide durable biocompatible coatings for medical devices intended for use in hemodynamic environments.

The present invention differs from all other prior art methods for making drug eluting polymeric coatings for medical devices. As mentioned briefly above, the use of block copolymers to make drug eluting coating is known in the art. See for example U.S. Pat. No. 6,855,770 B2 to Pinchuk et al issued on Feb. 15, 2005 (hereinafter the '770 patent). However, the polymers disclosed in the '770 patent are limited to block copolymers made using carbocationic living free radical polymerization. The reason that the '770 patent is limited to a very few specific polymer compositions is the inherent lack of controllability associated with ionic free radical polymerization, be it anionic or carbocationic. For example, in the '770 patent the inventors state that “[i]ₙ general, the polymerization reaction is conducted under conditions that minimize or avoid chain transfer termination of the growing polymer chains. Steps are taken to keep active hydrogen atoms (water, alcohol and like) to a minimum. The temperature of the polymerization is usually between −10° C. and −90° C., the preferred range being between −60° C. and −80° C., although lower temperatures may be employed if desired.” (Emphasis added) (See U.S. Pat. No. 6,855,770 B2 at column 5 line 62 through column 6 line 2). Thus, the prior art teaches a reaction that is subject to disproportionate termination, premature termination and must be conducted under extremely cold conditions, temperatures where many solvents are solids. Therefore, the prior art processes are very difficult to control and lack sufficient versatility to provide a useful range of block copolymers for controlled release coating applications.

It would be desirable then to provide methods that are more versatile, can be performed over a wider range of reaction conditions and can employ protonated, polar solvents to increase the number of monomer options. Moreover, the ability to control the reaction more closely would provide a means to stop; and restart polymerization, an option not available to prior art processes. However, before continuing with the details of the present invention a brief word on polymer chemistry is in order.

The term polymer is generally used to describe macromolecules comprising repeating units or “mers” (from the Greek word meros—part). Each individual repeating unit is connected to the other by covalent bonds and may form a variety of secondary structures. The simplest polymer comprises linear chains of randomly repeating units such as polyethylene as depicted in Formula 1 and the repeating unit of polyethylene, ethylene both depicted in Formula 1:

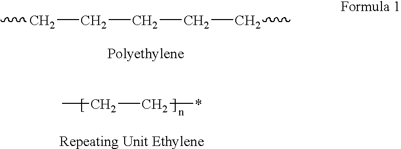

The repeating unit, ethylene, represents the intermolecular configuration for the repeating unit, it being understood that the terminal repeats will be different for valance requirements, for example —CH₂—CH₃. The “n” in Formula 1 represents the number of repeating units present in the polymer and is referred to as the “degree of polymerization” or DP; “n” can be any integer from 1 to 100 or more.

Alternatively, polymers can be branched rather than linear with the branches being of varying size and complexity. Branched polymers may also be star shaped or “dendritic,” combed shaped, ladder shaped or form complex networks when the branches become interconnected.

Polymers can be made up of either single repeating structural subunits as depicted in Formula 1 or from different repeating units. When two or more structural units comprise the polymer it is referred to as a copolymer such as ethyl methacrylate (EMA) and styrene as depicted below in Formula 2.

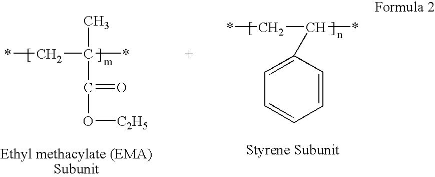

In Formula 3 below the individual subunits, EMA and styrene are organized in a regular repeating sequence alternating between EMA and styrene and thus is referred to as an alternating copolymer. However, the order can also be random and in this configuration would be referred to a random copolymer.

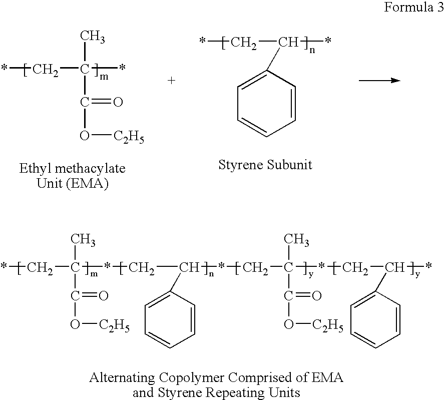

Additionally copolymers can comprise subunits that are neither random nor alternating but rather organized in an ordered sequence. These are commonly referred to as “block copolymers.” Block copolymers are made up of blocks of individual polymers joined by covalent bonds. For example, Formula 4 depicts a block copolymer of a polystyrene block and a polyisoprene block:

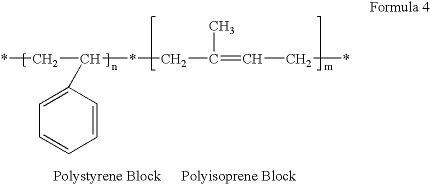

As indicated by the subscripted “n” and “m,” the blocks may vary in length and can vary in repeat sequence. For example, the polymer depicted in Formula 4 is a polystyrene, polyisoprene di-block. Traditionally, each polymer constituent is designated either “A” or “B.” For convenience polystyrene will be designated “A” and polyisoprene will be designated “B.” Thus the polystyrene-polyisoprene di-block of Formula 4 could assume one, or more, of the following configurations: -[AB][AB][AB][AB][AB][AB]-; [AA-BB]-[AA-BB]-[AA-BB]-; or -[AAAA-BBBB]-[AAAA-BBBB]-[AAA-BBBB]- etc.

A tri-block copolymer could include yet a third polymer block such as EMA and be designated “C” with each polymer block being covalently bound to the others. A typical tri-block would be -[AAA-BBB-CCC]-[AAA-BBB-CCC]- and so on. For convenience, each block polymer is depicted as a homopolymer, however, the individual blocks can also be copolymers. A tri-block can also comprise a regular repeating sequence of two subunits arranged in a specific order such as [AAA-BBB-AAA] where each subunit [AAA-BBB-AAA] is a “block.”

Some block copolymers are linear, in which the blocks are connected end-to-end; however, it is possible to form other types of block-copolymers including star copolymers (also known as dendritic copolymers), in which all of the blocks are connected via one of their ends at a single junction. More complicated arrangements are also possible. The number of monomer types in a block copolymer may be less than or equal to the number of blocks. Thus, an ABC linear tri-block consists of three monomer types, whereas an ABA linear tri-block consists of two monomer types.

Polymers can also have a backbone comprised of a single polymer chain such as polyethylene having another polymer extending from the back bone. Such polymers are commonly referred to as graft copolymers. An example of a typical graft copolymer, a graft copolymer of acrylonitrile with polyethylene, is depicted in Formula 5.

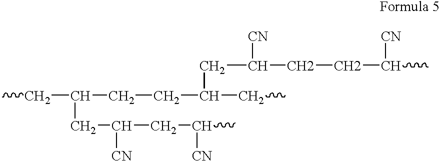

### A Typical Graft Copolymer

A noted above, in both block and graft copolymers the length of the uninterrupted sequences (A, B, or C for example) may vary.

It is appropriate to briefly discuss polymer nomenclature. The first efforts to categorize polymers were based on the reaction products and methods of formation. Two classes of polymers were initially recognized: condensation polymers and addition polymers. Condensation polymers refer to those polymers where certain atoms are lost from the monomer subunits as the polymer chain grows. For example, when polyamide is formed by “condensing” a diacid chloride with a diamine, hydrochloric acid is lost. The chloride being provided by the diacid chloride and the diamine contributing the hydrogen atom.

Addition polymers are polymers with identical structures of the repeat units to the monomers from which they are derived, generally with the exception of the loss of a carbon-carbon double bond. Polystyrene is an example of an addition polymer.

However, the two categories, condensation and addition polymers, failed to account for the diverse polymer types commonly seen today. Thus a superior definition has been proposed. In this alternate categorization scheme, polymers are classified by how monomer or block subunits are added to the growing chain. For example, polymers formed through reactions that occur in discrete steps are referred to as “step-growth” polymers and include condensation polymers. Step-growth polymers require long periods of time, usually measured in hours, for the macromolecule to form.

Another class of polymers are the so-called chain-growth polymers and are formed using chain propagation reactions and depend on an active center on the growing chain's end. These are highly reactive polymers that take mere seconds to form. Chain-growth polymer may proceeded via free radical polymerization or by ionic polymerization. This class is also referred to herein as “living polymerization.” It is this class of chain propagation that will be discussed further. It is important to in mind that prior art living polymer polymerization such as carbocation (as disclosed in the '770 patent) and conventional free radical polymerization differ significantly from the methods of the present invention (RAFT and ATRP) and it is this difference that the present inventors have surprisingly discovered leads to superior and more versatile drug-eluting polymer coatings than disclosed in the prior art.

Recently, polymers have been used with increasing frequency as drug-eluting reservoirs for coating medical devices. Polymeric drug-eluting polymers have proven useful in providing the controlled release of bioactive agents (drugs) in situ. However, in order to achieve precise control over the drug release profile many factors must be taken into consideration. For example, the solubility factors associated with the drug and polymer should be determined in order to avoid tedious and generally irreproducible trial and error. Moreover, once the solubility factors are known, it is possible for polymer scientists to modify a polymer's chemistry to meet specific needs. (See co-owned U.S. patent application Ser. No. 11/005,463 the entire contents of which are incorporated herein by reference).

One means for altering the drug elution profile of a polymer coating is to mix different polymer components in different ratios. For example, mixtures of different polymers and/or copolymers having differing hydrophilicities and hydrophobicities can significantly affect the coating's performance. However, polymer blends can be difficult to compatiblize and in some circumstances polymer blends can be non-uniform resulting in inconsistent drug elution profiles. Another method for tuning a polymer (as used herein polymer tuning refers to a process of adjusting a polymer's composition to achieve a desired drug elution profile and other physical charateristics) is to alter the individual monomers that comprise a given polymer. Thus polymer scientists have experimented using condensation and addition techniques to tune specific polymers. However, while condensation and addition techniques are useful with relative simple polymers, more complex polymer structures are difficult to achieve using these methods. This is especially true when polymers are used in biomedical applications where the multi-factorial demands on a polymer's performance are critical and the margin for error is essentially non-existent. Thus, for the reasons already described, the present inventors turned to block copolymers as a possible alternatives to polymer coatings derived from blending a limited number of miscible polymers and copolymers and/or being limited to the few existing block copolymers made using the teachings of the prior art such as those disclosed in the '770 patent. However, block copolymers seemed a promising alternative, but methods are needed that permitted the use of a wider range of monomer subunits, combinations of polymers and bioactive agents and more production friendly manufacturing techniques were needed. Therefore, the present inventors sought to develop new methods for making drug eluting polymer coatings.

In the past decade, significant efforts have been devoted to the development of controlled living polymerization based on the free radical chemistry. Three main approaches have been developed: the first involves the mediation of the controlled free radical procedure by stable free radical polymerization (SFRP), and the second, reversible addition-fragmentation transfer polymerization (RAFT), while the third, atom transfer radical polymerization (ATRP). Controlled radical polymerization is a very rapidly developing field as a result of its commercial importance, facile reaction conditions and synthetic manipulations, which are much easier than in anionic or carbocationic systems. It combines all the advantages of radical polymerization, including the ability to use a large range of monomers which can be polymerized and undemanding reaction conditions, including monomer purification, residual water, wide temperature range, and the use of bulk systems. In addition, it allows much easier preparation of random and block copolymers than most ionic reactions, due to closer reactivity ratios in radical systems. At the same time controlled radical polymerization includes the best features of living anionic and carbocationic systems, allowing the synthesis of polymers with predetermined molecular weights, low polydispersities, and end-group functionalities, as well as various possibilities for structural control, including chain architecture and composition. Therefore, the present inventors have now discovered that controlled living polymerization based on the free radical chemistry, specifically RAFT and ATRP, provide preferred methods for developing drug-eluting block copolymer implantable medical device coatings.

In one non-limiting examplary embodiment of the present invention (merely a schematic representation), a diblock copolymer useful for providing drug-eluting coatings for medical devices having the general formula of Formula 6 is provided. Note that the “block” as used in the following Formula refers to a linking group (such as DMDBH) between the individual constituent groups.

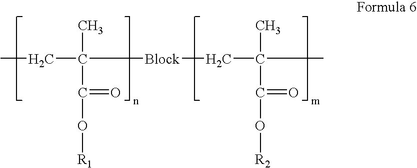

In Formula 6, R₁ and R₂ are independently a C₁-C₁₀ straight chain, branched, substituted or unsubstituted alkyl group, a C₁-C₁₀ straight chain, branched, substituted or unsubstituted alkenyl, a C₁-C₂₀ substituted or unsubstituted cyclic alky, a C₁-C₂₀ substituted or unsubstituted heterocyclic alkyl, an acyl group, an aryl group, an adamantyl group or an benzyl group; wherein said substitute group can be a halogen, sulphur, phosphorus, an amine, an amide, an imine, an imide, an alcohol, or alkoxy. Moreover, n and m are integers from 1 to 100 and independently denote the number of repeating units per block.

The block copolymers of the present invention can take the form of di-block linear copolymers, tri-block linear copolymers, multi-block linear copolymers and multi-arm star block copolymers constructed from two or more monomer units. In a non-limiting example of monomers suitable for constructing the block copolymers of the present invention, two of more monomers are selected from the group consisting of methacrylate, acrylate, styrene, N-vinyl pyrrolidone, vinyl acetate, vinyl ether and vinyl alcohol monomer units.

One embodiment of the present invention for synthesizing the block copolymers of the present invention is ATRP. Atom transfer radical polymerization is a relatively new approach to controlled radical polymerization involving the transfer of a halogen atom between a transition metal complex and the end of a polymer chain. Atom transfer radical polymerization is an example of controlled living radical polymerization and provides control over molecular weights, polydispersities, functionalities, chain composition and topologies previously unattainable with existing technologies. The ATRP reactions can be catalyzed by transition metal complexes including, but not limited to, Cu(I), Ru(II), Fe(II), Pd(II), Rh(III) and Re(II).

Reversible addition-fragmentation chain transfer (RAFT) is a second method for synthesizing the block copolymers of the present invention. This is a controlled free radical polymerization methodology that allows the synthetic tailoring of macromolecules with complex architectures, including block copolymers, with predetermined molecular weight, terminal functionality and narrow molecular weight distribution.

The following examples are provided to more precisely define and enable the medical device coatings and methods of the present invention. It is understood that there are numerous other embodiments and methods of using the present invention that will be apparent embodiments to those of ordinary skill in the art after having read and understood this specification and examples.

### EXAMPLE 1

### Synthesis of Block Copolymers using the ATRP Method

An exemplary tri-block copolymer was synthesized according to the method of Scheme 1 below. Specifically, 28.7 mg CuBr(I) and a magnetic stir bar were charged to a 60 mL bottle which was then sealed with a rubber septum and subjected to three cycles of vacuum/nitrogen. Fifteen milliliters of deoxygenated 2-butanone were then injected and the bottle and the vacuum/nitrogen cycles repeated. Then, 42 μL of pentamethyldiethylenetriamine were injected and the cycles of vacuum/nitrogen repeated before 8.0 mL of deoxygenated n-hexyl methacrylate was injected and the vacuum/nitrogen cycles repeated. Finally, 22 μL dimethyl-2,6-dibromoheptanedioate was injected. The living polymerization was carried out at 75° C. for 6 hours before 8.0 mL methyl methacrylate was added and the polymerization was then allowed to continue for 6 hours.

The resultant polymer was purified by three cycles of precipitation in methanol. The purified polymer had a number average molecular weight of 51800 and polydispersity indices (PDI) of 1.60 (gel permeation chromatography [GPC] in tetrahydrofuran [THF], polystyrene standard). The composition of the resultant tri-block copolymer was analyzed with ¹H nuclear magnetic resonance (NMR) and determine to be composed of 84% hexyl methacrylate units and 16% methyl methacrylate units.

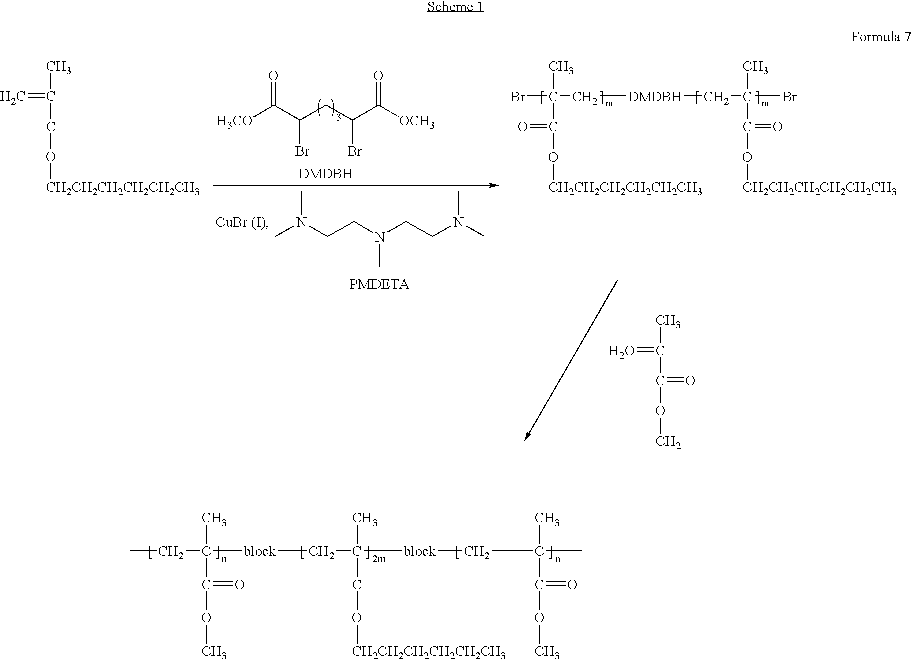

### EXAMPLES 2 THROUGH 4

### RAFT Synthesis Examples

In Examples 2 through 4 that follow, the block copolymers of the present invention are synthesized using the RAFT method as discussed above and depicted generally in Scheme 2. Specifically, in Examples 2 through 4 the RAFT agent used is depicted below as Formula 8.

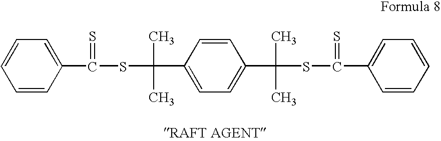

### EXAMPLE 2

### Synthesis of poly(hexyl methacrylate)

A bottle with a magnetic spin bar was charged with 5.0 g purified n-hexyl methacrylate (HMA, which was purified by passing through basic alumina column), 2 mL anisole, 3.5 mg of azobisisobutyronitrile (AIBN), and 93.4 mg of RAFT agent. The bottle was subjected to vacuum/nitrogen cycle 10 times. The bottle was heated in an oil bath at 60° C. for 88 hours. The polymer was purified by precipitation in methanol three times from acetone solution. The polymer has a number average molecular weight of 17600 and PDI of 1.07 (GPC in THF, universal calibration with polystyrene). The ¹H NMR spectrum is consistent the structure of poly(n-hexyl methacylate) end-capped with the RAFT functional groups.

### EXAMPLE 3

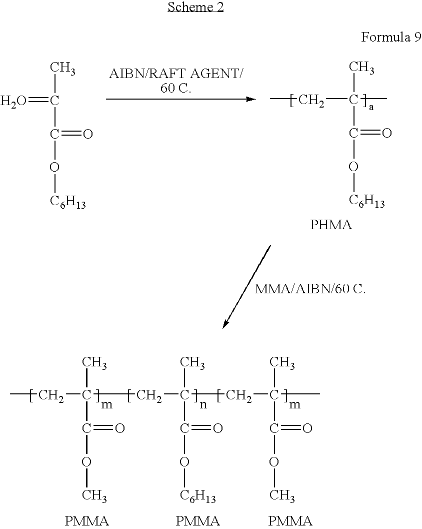

A bottle was charged with 300 mg of poly(n-hexyl methacrylate) from Example 3, 0.5 mg of AIBN, 0.75 mL of methyl methacrylate (MMA) and 0.25 mL of 1,4-dioxane. A magnetic spin bar was added and the bottle was sealed and subjected to vacuum/nitrogen cycle 10 times. The mixture was heated at 60° C. in an oil bath for 25 hours. The polymer was purified by precipitation in methanol three times from acetone solution. The composition of the block copolymer was analyzed with ¹H NMR. The tri-block copolymer was composed of 26% hexyl methacrylate units and 74% methyl methacryalte units. The polymer has a number average molecular weight of 39870 and PDI of 1.16 (GPC in THF, universal calibration polystyrene standard).

### EXAMPLE 4

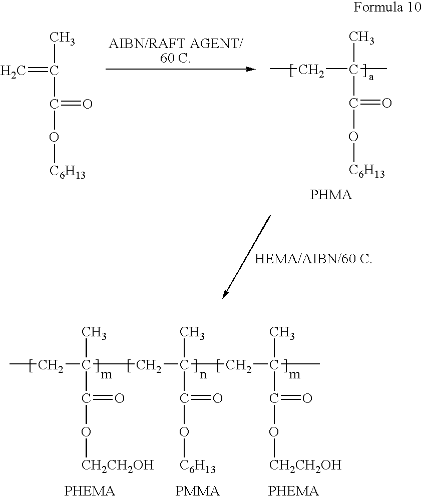

Poly(n-hexyl methacrylate) with higher molecular weight (Mn=65400, PDI=1.16) was similarly synthesized as in Example 3 with less amount of RAFT agent. A bottle was charged with 1.0 g of above higher molecular weight poly(n-hexyl methacrylate), 1 mL of 2-butanone, 1 mL of 1-propanol, 0.25 g of 2-hydroxyethyl methacylate (HEMA) and 1.1 mg of AIBN. The bottle was subjected to vacuum/nitrogen cycle 10 times. The mixture was heated at 60 C in an oil bath for 66 hours. The solvent and residual monomer was removed under high vacuum. The polymer is soluble in a mixed solvent of chloroform/methanol (v/v 50/50). The molecular weight was not determined. The composition of the block copolymer was analyzed with ¹H NMR. The triblock copolymer was composed of 83% hexyl methacrylate units and 17% 2-hydroxyethyl methacryalte units.

### EXAMPLE 5

### Coating of Stents with Block Copolymers

As previously discussed, the block copolymers of the present invention can be applied to virtually any medical device surface using standard coating techniques including spraying, dipping, or painting. In one embodiment of the present invention the block copolymer coatings are sprayed onto the surface of a vascular stent that has been previously provided with a parylene C primer coat. The parylene C having been applied first to the cleaned, bare stent surface using vacuum deposition.

Spraying is carried out in an isolator employing an ultrasonic spray device. The spray device's coating chamber is filled with a solution of the block copolymer of the present invention and programmed to deliver approximately 45 μg per mm of stent. In one embodiment of the present invention, 400 μg of block copolymer coating is loaded on a 9 mm stent. The stents are then mounted onto a mandrel and sprayed. After the spraying operation is complete the stent is dried under vacuum at room temperature overnight.

In one embodiment of the present invention the block copolymers of the present invention are useful for coating implantable medical devices such as vascular stents and stent grafts useful for the treatment, inhibition and prevention of restenosis. Vascular stents present a particularly unique challenge for the medical device coating scientist. Vascular stents (hereinafter referred to as “stents”) must be flexible, expandable, biocompatible and physically stable. Stents are used to relieve the symptoms associated with coronary artery disease caused by occlusion in one or more coronary artery. Occluded coronary arteries result in diminished blood flow to heart muscles causing ischemia induced angina and in severe cases myocardial infarcts and death. Stents are generally deployed using catheters having the stent attached to an inflatable balloon at the catheter's distal end. The catheter is inserted into an artery and guided to the deployment site. In many cases the catheter is inserted into the femoral artery or of the leg or carotid artery and the stent is deployed deep within the coronary vasculature at an occlusion site.

Once positioned at a treatment site the stent or stent graft is deployed, generally using balloon catheters. The balloon expands the stent gently compressing it against the arterial lumen clearing the vascular occlusion or stabilizing the plaque. The catheter is then removed and the stent remains in place permanently. Most patients return to a normal life following a suitable recovery period and have no reoccurrence of coronary artery disease associated with the stented occlusion. However, in some cases the arterial wall's initma is damaged either by the disease process itself or as the result of stent deployment. This injury initiates a complex biological response culminating is vascular smooth muscle cell hyperproliferation and occlusion, or restenosis, at the stent site.

Recently significant efforts have been devoted to preventing restenosis. Several techniques including brachytherapy, excimer laser, and pharmacological techniques have been developed. The least invasive and most promising treatment modality is the pharmacological approach. A preferred pharmacological approach involves the site-specific delivery of anti-proliferative drugs directly to the stent deployment area. Site specific delivery is preferred over systemic delivery for several reasons. First, many anti-proliferative drugs are highly toxic and cannot be administered systemically at concentrations needed to prevent restenosis. Moreover, the systemic administration of drugs can have unintended side effects at body locations remote from the treatment site. Additionally, many drugs are either not sufficiently soluble, or too quickly cleared from the blood stream to effectively prevent restenosis. Therefore, administration of anti-restenotic compounds directly to the treatment area is preferred.

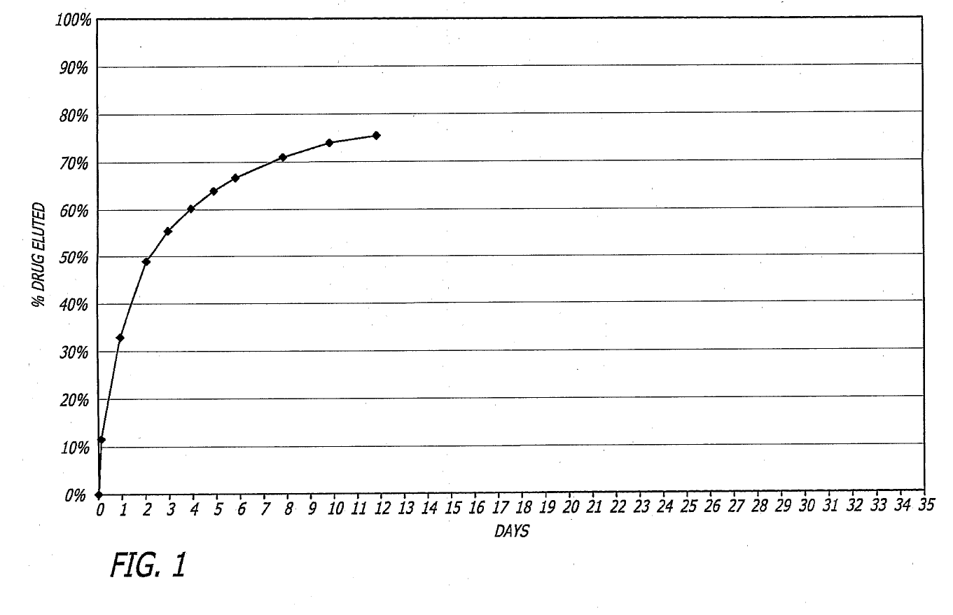

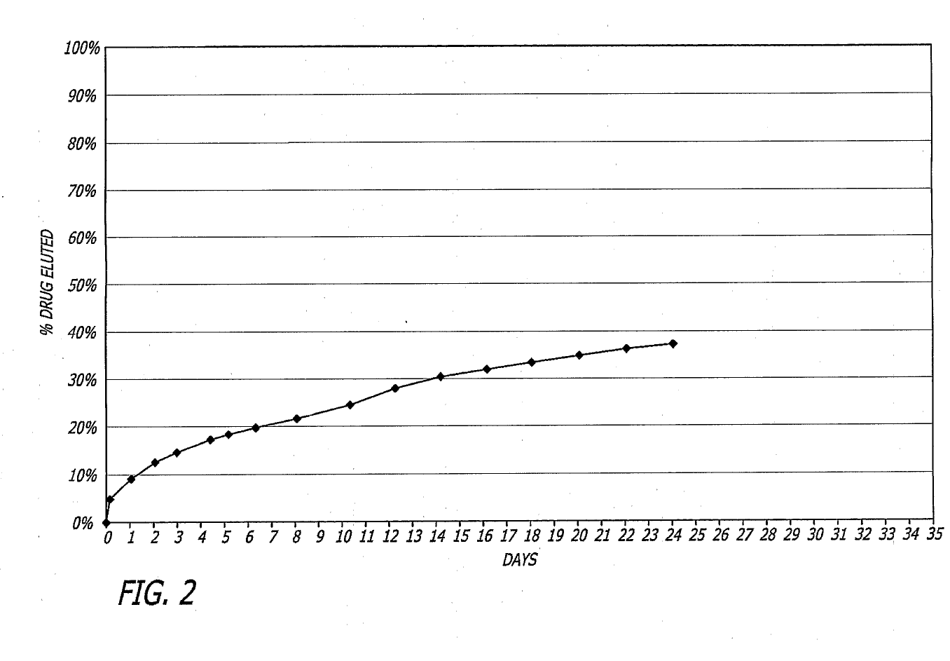

The most successful method for localized drug delivery developed to date is the drug-eluting stent. Many drug-eluting stent embodiments have been developed and tested. However, significant advances are still necessary in order to provide safe and highly effective drug delivery stents. One of the major challenges associated with stent-based drug delivery is controlling the drug delivery rate. Generally speaking drug delivery rates have two primary kinetic profiles. Drugs that reach the blood stream or tissue immediately after administration follow first-order kinetics. First-order drug release kinetics, as depicted in FIG. 1 provide an immediate surge in blood or local tissue drug levels (peak levels) followed by a gradual decline (trough levels). In most cases therapeutic levels are only maintained for a few hours. Drugs released slowly over a sustained time where blood or tissue concentrations remains steady follow zero-order kinetics as depicted in FIG. 2. Depending on the method of drug delivery and tissue/blood clearance rates, zero-order kinetics result in sustained therapeutic levels for prolonged periods. Drug-release profiles can be modified to meet specific applications. Generally, most controlled release compositions are designed to provide near zero-order kinetics. However, there may be applications where an initial burst, or loading dose, of drug is desired (first-order kinetics) followed by a more gradual sustained drug release (near zero-order kinetics).

Another application for coated medical devices of the present invention include vulnerable plaque stabilization. Vulnerable plaque is composed of a thin fibrous cap covering a liquid-like core composed of an atheromatous gruel. The exact composition of mature atherosclerotic plaques varies considerably and the factors that affect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type. When atherosclerotic plaque is prone to rupture due to instability it is referred to a “vulnerable” plaque. Upon rupture the atheromatous gruel is released into the blood stream and induces a massive thrombogenic response leading to sudden coronary death. Recently, it has been postulated that vulnerable plaque can be stabilized by stenting the plaque. Moreover, vascular stents having a drug-releasing coating composed of matrix metalloproteinase inhibitor dispersed in, or coated with (or both) a polymer may further stabilize the plaque and eventually lead to complete healing.

Treatment of aneurysms is yet another application for the drug-eluting stents of the present invention. An aneurysm is a bulging or ballooning of a blood vessel usually caused by atherosclerosis. Aneurysms occur most often in the abdominal portion of the aorta. At least 15,000 Americans die each year from ruptured abdominal aneurysms. Back and abdominal pain, both symptoms of an abdominal aortic aneurysm, often do not appear until the aneurysm is about to rupture, a condition that is usually fatal. Stent grafting has recently emerged as an alternative to the standard invasive surgery. A vascular graft containing a stent (stent graft) is placed within the artery at the site of the aneurysm and acts as a barrier between the blood and the weakened wall of the artery, thereby decreasing the pressure on artery. The less invasive approach of stent-grafting aneurysms decreases the morbidity seen with conventional aneurysm repair. Additionally, patients whose multiple medical comorbidities make them excessively high risk for conventional aneurysm repair are candidates for stent-grafting. Stent grafting has also emerged as a new treatment for a related condition, acute blunt aortic injury, where trauma causes damage to the artery.

Regardless of the clinical application, the present invention is directed at optimized drug-releasing medical device coatings suitable for use in hemodynamic environments. The coatings of the present invention are composed of polymers having at least one drug composition dispersed therein. The polymeric compostions of the present invention have been specifically formulated to provide medical device coatings that tenaciously adhere to medical device surfaces (do not delaminate), flex without fracturing (ductile), resist erosion (durable), are biocompatible and release a wide variety of drugs at controlled rates.

Polymers have been used as medical device coatings for decades to enhanced biocompatibility and erosion resistance. Moreover, in certain applications polymer coatings may also provide electrical insulation. It is also well known in the art that polymers can act as reservoirs and/or diffusion barriers to control biological agent elution rates.

Recently, coatings have been applied to implantable medical devices such vascular stents, vascular stent grafts, urethral stents, bile duct stents, catheters, inflation catheters, injection catheters, guide wires, pace maker leads, ventricular assist devices, and prosthetic heart valves. Devices such as these are generally subjected to flexion strain and stress during implantation, application or both. Providing flexible medical devices such as stents with stable biocompatible polymer coatings is especially difficult.

There are two basic molecular morphologies that define a polymer's tertiary solid-state structure. Polymers may be either semi-crystalline or amorphous depending on the nature of the polymer subunit. Semi-crystalline polymers are ridged and brittle at any temperature below their melting point and are generally not suitable for coating flexible medical devices such as stents. In addition, drugs or bioactive agents can not stay in the polymer crystal region, therefore, the drugs or bioactive agents loading is limited. Amorphous polymers, on other hand, can be either rigid or elasticity/ductile depending on its glass transition point (Tg). The Tg of an amorphous polymer is the temperature above which the amorphous polymer is elastic/ductile and flexible. For stent applications it is desirable that the Tg be below body temperature. Many polymeric compositions have Tgs substantially above body temperature and are thus in the glassy or rigid state when the device is deployed and remains so once the device is implanted. Polymers in the “glassy” state are non-elastic/ductile and prone to cracking, fracturing and delaminating when the stent is flexed. Polymer coatings susceptible to fracture and delaminating are especially undesirable when used on stents. Small polymer particles that separate from a delaminated or fractured stent coating may be carried by the blood flow downstream where they can lodge in capillaries and obstruct blood flow to critical regions of the heart. Therefore stents and other flexible medical devices should have polymer coatings that are elastic/ductile and adhere to the device surface well. Generally, this requires that coating polymers be amorphous and have Tgs below body temperature.

However, polymers having extremely low Tgs are undesirable when used to coat devices that are subjected to continual hemodynamic forces. As general rule, the lower the Tg the more rubbery a polymer becomes. More rubbery polymers can be tacky and less durable and are more likely to break down when exposed to hemodynamic induced stress and wear than less rubbery ones. This is partially due to the fact that the more rubbery polymers have higher coefficients of friction and possess less structural integrity. Therefore, polymers having extremely low Tgs should not be the dominant polymer in polymer blends or copolymer compositions when designing coating polymers intended for stents and other vascular implants. In addition, extremely low Tg (e.g., rubbery) polymers tend to release drugs or bioactive materials at undesirably fast rates due to their high free volumes.

In addition to the aforementioned structural and drug-releasing profile considerations, polymers used as stent coatings must also be biocompatible. Biocompatibility encompasses numerous factors that have been briefly defined in the preceding “Definition of Terms” section. The need for a polymer to be biocompatible significantly limits the number of available options for the material scientist. Moreover, these options are further limited when the polymer coating is used on a device that is continuously exposed to hemodynamic forces. For example, stent coatings must remain non-thrombogenic, non-inflammatory and structurally stable for prolong time periods.

There are generally two large, and to some extent overlapping, categories of biocompatible polymers suitable as medical device coatings: bioerodable (including bioresorbable polymers) and non-bioerodable polymers. Coating compositions of the present invention are principally directed at the latter. However, the present invention's methods are equally applicable to biorerodable and non-bioerodable polymer coatings. The remaining discussion and exemplary embodiments will be directed at non-bioerodable polymers.

Non-bioerodable polymers can be hydrophilic, hydrophobic or amphiphilic depending on the polarity of the monomers or blocks used and the ratio of hydrophobic to hydrophilic monomers. Hydrophilic polymers are polar molecules that are miscible with polar solvents and are generally lubricious while contacting body fluids. Hydrophilic polymers are often used in biomedical applications to produce lubricious hydrogels. Hydrogels include polymer compositions that can absorb more than 20% of its weight in water while maintaining a distinct three-dimensional structure. This definition includes dry polymers that swell in aqueous environments in addition to the water-swollen polymer compositions.

Hydrophobic polymers such as polytetrafluoroethylene (PTFE, Teflon®) do not swell but can also be biocompatible. Teflon® has an extremely low coefficient of friction and is one of the most widely used hydrophobic biocompatible polymers. However, PTFE's slipperiness makes it difficult to handle and manipulate. Moreover, PTFE is a stiff chemically inert polymer and bonds poorly to surfaces. Furthermore, PTFE's extremely hydrophobic nature significantly limits its chemical compatibility with many bioactive agents. Recently, nanoporous PTFE has been developed that can be used as a barrier coating, or cap coat, that mediates bioactive agent release from an underlying drug reservoir (Advanced Surface Engineering, Inc. Eldersburg, Md.). However, nanoporous PTFE coatings are expensive and the application process is not compatible with all medical device surfaces and drug categories. Consequently, the usefulness of PTFE as a medical device coating is limited. There are many other biocompatible hydrophobic polymers; however, many of these have a high coefficient of frictions which is undesirable in a hemodynamic environment. Moreover, many hydrophilic drugs do not disperse well in hydrophobic polymer and therefore are not suitable drug delivery platforms for many hydrophilic bioactive agents.

Therefore, there are four specific attributes that the stent coating polymers made in accordance with the teachings of the present invention should possess. The polymer compositions of the present invention should be biocompatible, durable, elastic/ductile and possess a predetermined drug release profile. Other requirements include processing compatibility such as inert to ethylene oxide (ETO) sterilization.

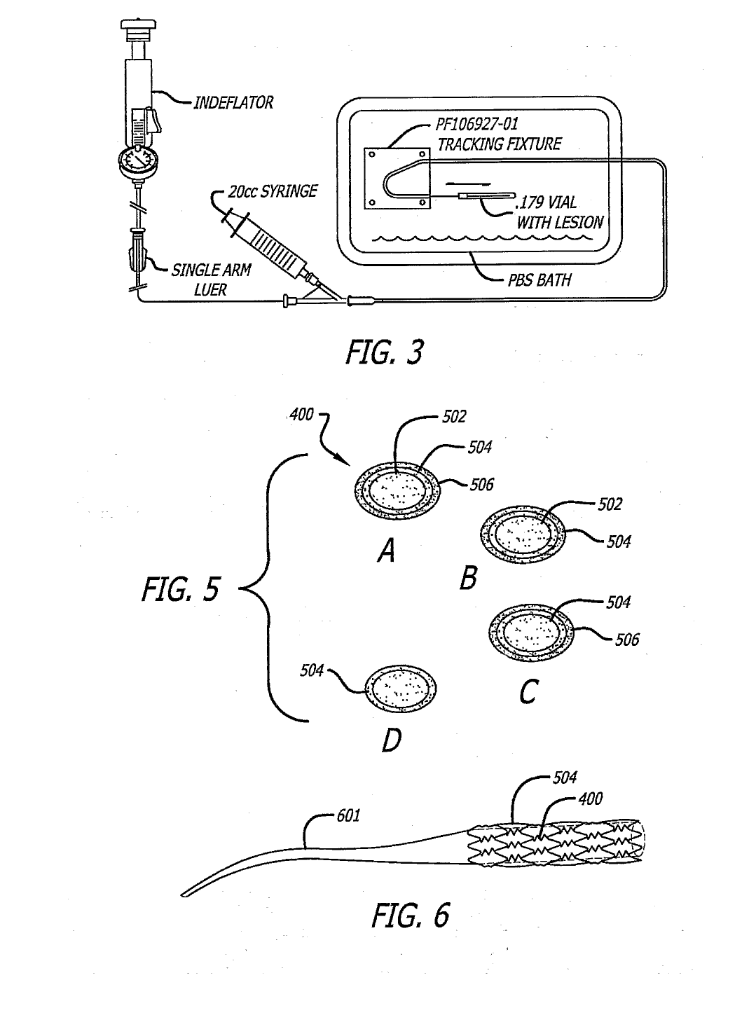

A copolymer's biocompatibility, elasticity/ductility and durability can be optimized by altering the ratio of polymeric subunits that favor one property over another. For example, ductility and durability are roughly a function of the polymer's Tg. The lower the Tg, the more ductile the polymer becomes (see FIG. 3 for an example of a suitable testing device/method for assessing ductility and durability). However, below a certain point the polymer becomes too rubbery and its durability is adversely affected. Moreover, extremely rubbery polymers possess greater first-order kinetics than near zero-order kinetics, consequently, extremely low Tgs are to be avoided.

Release rate is not entirely a function of drug-polymer compatibility. Coating configurations, polymer swellability and coating thickness also play roles. When the medical device of the present invention is used in the vasculature, the coating dimensions are generally measured in micrometers (μm). Coatings consistent with the teachings of the present invention may be a thin as 1 μm or a thick as 1000 μm. There are at least two distinct coating configurations within the scope of the present invention. In one embodiment of the present invention the drug-containing coating is applied directly to the device surface or onto a polymer primer coat such a parylene or a parylene derivative. Depending on the solubility rate and profile desired, the drug is either entirely soluble within the polymer matrix, or evenly dispersed throughout. The drug concentration present in the polymer matrix ranges from 0.1% by weight to 80% by weight. In either event, it is most desirable to have as homogenous of a coating composition as possible. This particular configuration is commonly referred to as a drug-polymer matrix.

Drugs suitable for use in the controlled release block copolymer coatings of the present invention include bioactive agents including, but not limited to, macrolide antibiotics, estrogens, chaperone inhibitors, protease inhibitors, protein-tyrosine kinase inhibitors, peroxisome proliferator-activated receptor gamma ligands, hypothemycin, nitric oxide, bisphosphonates, anti-proliferatives, paclitaxel, epidermal growth factor inhibitors, antibodies, proteasome inhibitors, antibiotics, anti-inflammatories, anti-sense nucleotides, transforming nucleic acids and protease inhibitors.

More specifically, drugs suitable for use in the controlled release coatings of the present invention include bioactive agents including, but not limited to, rapamycin and analogues thereof, paclitaxol and analogs thereof, actinomycin-D and analogs thereof, zotarolimus, everolimus, 17AAG, tempostatin, xemilofiban, cilostazol, vinblastine, epothalone-D, combretastatin A4, A2A agonists, leptomycin B, fumagillin, TNP-470, ICP-2, brifeldin A, homoharringtonone and campothecin.

We now turn to another factor that contributes to the controlled release block copolymer coatings of the present invention. As mentioned earlier, coating intended for medical devices deployed in a hemodynamic environment must possess excellent adhesive properties. That is, the coating must be stably linked to the medical device surface. Many different materials can be used to fabricate the implantable medical devices including stainless steel, nitinol, aluminum, chromium, titanium, ceramics, and a wide range of plastics and natural materials including collagen, fibrin and plant fibers. All of these materials, and others, may be used with the controlled release coatings made in accordance with the teachings of the present invention.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface. In one embodiment of the present invention medical devices, specifically stents, are provided with polymer primer coats that provide inert adhesion layers for the controlled release coatings of the present invention. Primer coatings suitable for use with the controlled release block copolymer coatings of the present invention include, but are not limited to, parylene C (also known as para-mono-chloro-paraxyxylene), parylene N (poly-para-xyxylene), phenoxy, polyamide, epoxy, polyacrylate and polymethacrylate. For example, and not intended as a limitation, parylene C is applied to the stent surface using vapor deposition techniques. Parylene is a hydrophobic, biocompatible, lubricious polymer that is transparent, flexible and meets USP class VI plastic requirements. Moreover, parylene is a gas-phase polymerized composition that completely forms to device surface topologies leaving a thin, pinhole-free base coat that is readily coated with other polymers. Parylene's hydrophobic nature can present challenges to coating scientists. However, when used in accordance with the teaching of the present invention, controlled release polymer compositions can be optimized to assure good long-term adhesion to the primer coat.

The controlled release block copolymer coatings of the present invention can be applied to medical device surfaces, either primed or bare, in any manner known to those skilled in the art. Applications methods compatible with the present invention include, but are not limited to, spray coating, electrostatic spray coating, plasma coating, dip coating, spin coating and electrochemical coating. Moreover, the controlled release coatings of the present invention may be used with a cap coat. A cap coat as used here refers to the outermost coating layer applied over another coating. In an exemplary embodiment, a drug-releasing block copolymer coating of the present invention is applied over a primer-coated medical device. Over the copolymer a polymer cap coat is applied. The cap coat may optionally serve as a diffusion barrier to further control the drug release, or provide a separate drug. The cap coat may be merely a biocompatible polymer applied to the surface of the sent to protect the stent and have no effect on elusion rates.

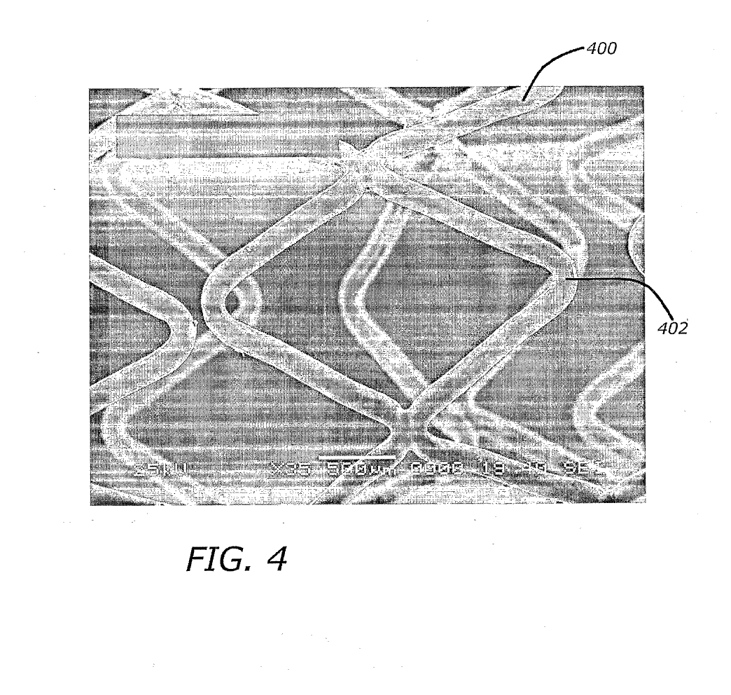

One embodiment of the present invention is depicted in FIG. 4. In FIG. 4 a vascular stent 400 having the structure 402 is made from a material selected from the non-limiting group materials including stainless steel, nitinol, aluminum, chromium, titanium, ceramics, and a wide range of plastics and natural materials including collagen, fibrin and plant fibers. The structure 402 is provided with a coating composition made in accordance with the teachings of the present invention. FIG. 5a-d are cross-sections of stent 400 showing various coating configurations. In FIG. 5a stent 400 has a first polymer coating 502 comprising a medical grade primer, such as but not limited to parylene or a parylene derivative; a second controlled release coating 504; and a third barrier, or cap, coat 506. In FIG. 5b stent 400 has a first polymer coating 502 comprising a medical grade primer, such as but not limited to parylene or a parylene derivative, and a second controlled release coating 504. In FIG. 5c stent 400 has a first controlled release coating 504 and a second barrier, or cap, coat 506. In FIG. 5 d stent 400 has only a controlled release coating 504. FIG. 6 depicts a vascular stent 400 having a coating 504 made in accordance with the teachings of the present invention mounted on a balloon catheter 601.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A medical device having a controlled release coating comprising a block copolymer wherein said block copolymer is selected from the group consisting of di-block linear copolymers, tri-block linear copolymers, multi-block linear copolymers and multi-arm star block copolymers and wherein said block copolymer is made using atom transfer radical polymerization (ATRP) or reversible addition-fragmentation chain transfer (RAFT).

2. The medical device of claim 1 wherein said block copolymer is a di-block having the general formula of Formula 6: 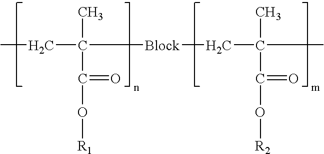
wherein R₁ and R₂ are independently a C₁-C₁₀ straight chain, branched, substituted or unsubstituted alkyl group, a C₁-C₁₀ straight chain, branched, substituted or unsubstituted alkenyl, a C₁-C₂₀ substituted or unsubstituted cyclic alky, a C₁-C₂₀ substituted or unsubstituted heterocyclic alkyl, an acyl group, an aryl group, an adamantyl group or an benzyl group; wherein said substitute group can be a halogen, sulphur, phosphosus an amine, an amide, an imine, an imide, an alcohol, or alkoxy; and wherein n and m are independently integers from 1 to 100.

3. The medical device of claim 2 wherein said block copolymer comprises at least one polymer block selected from the group consisting of elastomers, thermoplastic elastomers and thermoplastics.

4. The medical device according to claim 3 wherein said polymer blocks are selected from the group consisting of methacrylates, acrylates, styrene, N-vinyl pyrrolidone, vinyl acetate, vinyl ether, vinyl alcohol and combinations thereof.

5. The medical device according to claim 2 wherein R₁ and R₂ are the same, or independently methyl, ethyl, butyl, pentyl, hexyl, heptyl, octyl, dodecyl, polyether, cyclohexyl, cyclopentyl, cyclobutyl, norbutyl, benzyl, phenyl, adamantly, 2-hydroxylethyl, hydroxypropyl, hydroxybutyl, hydroxypentyl, hydroxyhexyl, choline, polytetrahydrofuran, and poly(propanol).

6. The medical device of claim 2 wherein said block copolymer is a thermoplastic elastomer and is comprised of a hard block a and a soft block b.

7. The medical device of claim 6 wherein said hard block has a glass transition temperature above 25° C. and said soft block has a glass transition temperature below 25° C.

8. The medical device of claim 7 wherein said hard block has a glass transition temperature between approximately 25° C. and approximately 50° and said soft block has a glass transition temperature between approximately −10° and approximately 25° C.

9. The medical device of claim 1 wherein said block copolymer comprises monomer units selected from the group consisting of methacrylate, acrylate, styrene, N-vinyl pyrrolidone, vinyl acetate, vinyl ether and vinyl alcohol.

10. The medical device of claim 2 wherein said block copolymer comprises the block copolymer of Formula 6 wherein said hard block R1 is hexyl methacrylate and said soft block R2 is methyl methacrylate.

11. The medical device of claim 1 wherein said block copolymer further comprises a bioactive agent.

12. The medical device of claim 11 wherein said bioactive agent is selected from the group consisting of macrolide antibiotics, estrogens, chaperone inhibitors, protease inhibitors, protein-tyrosine kinase inhibitors, peroxisome proliferator-activated receptor gamma ligands, hypothemycin, nitric oxide, bisphosphonates, anti-proliferatives, paclitaxel, epidermal growth factor inhibitors, antibodies, proteasome inhibitors, antibiotics, anti-inflammatories, anti-sense nucleotides, transforming nucleic acids and protease inhibitors.

13. The medical device of claim 12 wherein said bioactive agent is selected from the group consisting of rapamycin and analogues thereof, paclitaxol and analogs thereof, actinomycin-D and analogs thereof, zotarolimus, everolimus, 17AAG, tempostatin, xemilofiban, cilostazol, vinblastine, epothalone-D, combretastatin A4, A2A agonists, leptomycin B, fumagillin, TNP-470, ICP-2, brifeldin A, homoharringtonone and campothecin.

14. The medical device of claim 1 wherein said medical device is a vascular stent or stent graft.

15. The medical device of claim 1 further comprising a primer coat selected from the group consisting of parylene C, phenoxy, polyamide, epoxy, polyacrylate and polymethacrylate.

16. The method for preparing a medical device having a controlled release block copolymer coating containing a bioactive agent comprising:
depositing a solution of said block copolymer and said bioactive agent onto said medical device;
drying said medical device; and
annealing said medical device.

17. The method of claim 16 wherein said act of depositing is selected from the group consisting of spray coating, electrostatic spray coating, plasma coating, dip coating, spin coating and electrochemical coating.

18. The method of claim 16 wherein said medical device is a vascular stent.

19. The method of claim 16 wherein said medical device is a stent graft.

20. The method of claim 16 wherein said medical device further comprises a primer coat selected from the group consisting of parylene C, phenoxy, polyamide, epoxy, polyacrylate and polymethacrylate.

21. The method of claim 16 wherein said medical device further comprises a cap coat.

22. The method of claim 16 wherein said bioactive agent is an effective amount of an anti-restenotic drug.

23. The method of claim 22 wherein said anti-restenotic drug is selected from the group consisting of rapamycin and analogues thereof, paclitaxol and analogs thereof, actinomycin-D and analogs thereof, zotarolimus, everolimus, 17AAG, tempostatin, xemilofiban, cilostazol, vinblastine, epothalone-D, combretastatin A4, A2A agonists, leptomycin B, fumagillin, TNP-470, ICP-2, brifeldin A, homoharringtonone and campothecin.

24. The medical device of claim 16 wherein said medical device is delivered to the treatment site of a mammal in need thereof.

25. A method for treating vascular disease in a mammal in need thereof comprising delivering to a treatment site within a blood vessel a medical device having a block copolymer coating and an anti-restenotic drug for release of an effective amount of an anti-restenotic drug.

26. The method of claim 25 further comprising using a balloon catheter to place said stent or stent graft at said treatment site within said vessel.

27. The method of claim 25 wherein said vascular disease is selected from the group consisting of restenosis, vulnerable plaque and aneurysms.

28. A medical device having a controlled release coating comprising a tri-block copolymer wherein said tri-block copolymer is made using atom transfer radical polymerization (ATRP) and comprises Formula 8: 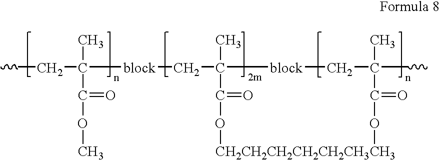
and wherein m and n are independently integers from 1 to 100.

29. A medical device having a controlled release coating comprising a tri-block copolymer wherein said tri-block copolymer is made using reversible addition-fragmentation chain transfer (RAFT) and comprises Formula 9: 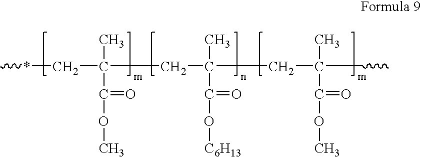
and wherein m and n are independently integers from 1 to 100.

30. A medical device having a controlled release coating comprising a tri-block copolymer wherein said tri-block copolymer is made using reversible addition-fragmentation chain transfer (RAFT) and comprises Formula 10: 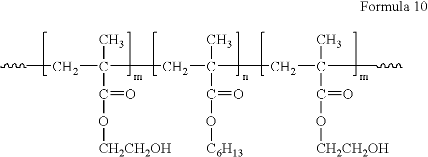
and wherein m and n are independently integers from 1 to 100.
